(19) United States
(12) Patent Application Publication
(10) Pub. No.: US 2015/0112109 A1
(43) Pub. Date: Apr. 23, 2015

(54) MULTIMETALLIC MIXED OXIDES, ITS PREPARATION AND USE FOR THE OXIDATIVE DEHYDROGENATION OF ETHANE FOR PRODUCING ETHYLENE

(71) Applicants: Instituto Mexicano del Petroleo, Mexico City (MX); PEMEX Petroquimica, Coatzacoalcos (MX); Universidad Politecnica de Valencia, Valencia (ES)

(72) Inventors: Jaime SANCHEZ VALENTE, Mexico City (MX); Enelio TORRES GARCIA, Mexico City (MX); Hector ARMENDARIZ HERRERA, Mexico City (MX); Maria de Lourdes Alejandra GUZMAN CASTILLO, Mexico City (MX); Andrea RODRIGUEZ HERNANDEZ, Mexico City (MX); Roberto QUINTANA SOLORZANO, Mexico City (MX); Maiby VALLE ORTA, Mexico City (MX); Jose Manuel LOPEZ NIETO, Valencia (ES)

(21) Appl. No.: 14/581,129

(22) Filed: Dec. 23, 2014

Related U.S. Application Data
(62) Division of application No. 13/835,490, filed on Mar. 15, 2013.

(51) Int. Cl. B01J 23/68; C07C 5/42; C07C 5/48; B01J 23/28

(52) U.S. Cl. CPC B01J 23/686; B01J 23/28; C07C 5/42; C07C 5/48; C07C 2521/08; C07C 2523/28; C07C 2523/68
USPC 585/658; 502/307; 502/312; 502/243; 585/656

(57) ABSTRACT

A layered multimetallic mixed oxide (LMMO) is characterized by one or more diffraction peaks at 5<2θ<15, preferably between 10<2θ<15. The catalysts can be represented by the general formula:

M1 M2 M3 O_{δ}
wherein
   M1 is selected from the group of Ag, Au, Zn, Sn, Rh, Pd, Pt, Cu, Ni, Fe, Co, an alkaline metal, an alkaline earth metal, a rare earth metal, or mixtures thereof.
   M2 is selected from the group of Ti, Hf, Zr, Sn, Bi, Sb, V, Nb, Ta and P, or mixtures thereof.
   M3 is selected from the group of Mo, W and Cr, or mixtures thereof.
δ depends on the amount and oxidation state or valence of the other components, also it depends on the starting materials, preparation method and the activation process, and where the catalyst exhibits at least one X-ray diffraction peak between 5<2θ<15.

### FIELD OF THE INVENTION

The present invention relates to layered multimetallic mixed oxides (LMMO) preparations, which present a layered structure characterized by diffraction peak or peaks between 5<2θ<15, preferably between 10<2θ<15. The presence of additional diffraction peaks is often observed, indicating the existence of extra crystalline phases; some crystalline arrangements were identified as monoclinic lattice of Silver Vanadium Molybdenum Oxide (ICDD-PDF 04-002-4830) or Cesium Vanadium Molybdenum Oxide (ICDD-PDF 00-030-0381) or monoclinic Sodium Vanadium Molybdenum Oxide (ICDD-PDF 04-011-9693) or monoclinic Lithium Vanadium Molybdenum Oxide (ICDD-PDF 04-006-7234) or orthorhombic Calcium Vanadium Molybdenum Oxide (ICDD-PDF 04-013-4035), among others crystalline structures. This invention also relates to the use of the LMMO in the partial oxidation of light hydrocarbons and particularly for the oxidative dehydrogenation of low molecular weight paraffins, preferably between 2 and 4 carbon atoms. More particularly the invention refers to the oxidative dehydrogenation of ethane to ethylene.

The LMMO catalysts of the present invention can be represented by the general formula:

M1 M2 M3 O_{δ}

### Wherein

M1 is selected from the group of Ag, Au, Zn, Sn, Rh, Pd, Pt, Cu, Ni, Fe, Co, an alkaline metal, an alkaline earth metal, a rare earth, or mixtures thereof.
M2 is selected from the group of Ti, Hf, Zr, Sn, Bi, Sb, V, Nb, Ta and P, or mixtures thereof.
M3 is selected from the group of Mo, W and Cr, or mixtures thereof.
δ depends on the amount and oxidation state or valence of the other components, also it depends on the starting materials, preparation method and the activation process.
   and where the catalyst exhibits at least one X-ray diffraction peak between 5<2θ<15.

### BACKGROUND OF THE INVENTION

Ethylene is one of the most important products produced in the petrochemical industry. It is used as raw material for the production of compounds of great importance for the chemical industry such as, polyethylene, styrene and polystyrene, among others. At present, most of the ethylene commercialized in the world is obtained by thermal cracking (pyrolysis) of several hydrocarbons fractions, including gas oil, naphtha and ethane. These processes are performed in the presence of superheated steam at high reaction temperatures, typically between 800 and 1000° C. Hence, under these conditions this process involves a significant demand of energy and produce a large diversity of by-products. By-products such as diolefins and acetylene are complex to remove. Usually, the separation of these two species of the reactor effluent, employ extractive distillation and/or a selective hydrogenation for the acetylene removal.

Consequently, for economic and environmental reasons, catalytic oxidative dehydrogenation of ethane (ODH-Et) is envisaged as a promising alternative for industrial ethylene production that exhibits clear advantages compared with aforementioned processes. ODH-Et, (i) is an exothermic reaction (ΔH°_{R,298K}=−106 kJ/mol); (ii) does not need a catalyst regeneration step because of the incorporation of oxygen in the feed, and (iii) is performed at relatively low reaction temperatures (<600° C.). This allows a considerable energy saving with the corresponding environmental benefits, by virtue of the decrease of CO₂ emissions to the atmosphere. Additionally, working at moderate temperatures has the advantage of limiting the number of side reactions and, thus, the associated by-products. In the case of ODH-Et, the more important byproducts are carbon monoxide and carbon dioxide, while the coke formation is negligible due to the use of an oxidant as part of the reaction mixture. In spite of the multiple efforts in the focused investigations for improving the activity and selectivity of catalytic systems for ODH-Et reaction, to date, an industrial application of ODH-Et is still far from a reality. Economic estimates by companies and/or research groups indicate that ethylene yields reported so far in literature are not attractive enough to consider the ODH-Et as an economically viable process. Therefore, many efforts are needed to meet the required catalytic performance, since, the production of carbon oxides, formed via very exothermic reactions, has to be minimized.

In this sense, molybdenum-vanadium based metal oxides are, among the mixed oxide systems, the most frequently used as active components of a catalyst for oxidation reactions. One of the main advantages of Mo and V cations is that they can be combined in a great number of crystalline structures with different oxidation states. The catalytic performance depends on the crystalline phases present, which could be related to individual molybdenum or vanadium oxides and/or to different crystalline arrangements where Mo and V are combined in several molar ratios. The redox properties of the catalyst are also tailored by the inclusion of other metal cations acting as promoters or inhibitors. Consequently, many catalytic systems have been proposed for this end, since, several active structures have shown notable partial oxidation performance. The structures of mixed oxides compounds are formed using the same metal cations.

Layered mixed oxides are a class of materials that are naturally occurring. They are interesting because the layers can be easily tuned by the chemical composition. Isomorphic substitution of cations is a usual way to reach this goal. Thus, depending of the nature of the cations building their layers, their physicochemical features are modulated. Therefore, compounds with layered structure based on molybdenum and vanadium cations have been proposed for partial oxidation. The addition of others metals is often employed to control their catalytic properties and/or their thermal stability.

Among the layered mixed oxides is the brannerite material (UTiO₂O₆), which presents a monoclinic crystal arrangement with main diffraction peaks, located at 2θ: 14.61, 18.65, 25.79, 26.53, 30.78 (ICSD #201342). A detailed description of this structure can be found in the following references: Ruth R. and Wadsley A. D. Acta Crystallogr. 21 (1996) 974; Galy J., Darriet J. and Darriet B. C. R. Acad. Sci. Paris Ser. C 264 (1967)1477; Galy J., Meunier J., Sengas J. and Hagenmuller P., J. Inorg. Nucl. Chem., 33 (1971) 2403; Ng H. N. and Calvo C. Canad. J. Chem. 50 (1972) 3619; Kozlowski R., Ziolkowski J., Mocala K. and Haber J. J. of Solid State Chem. 35, (1980)1, which are hereby incorporated by reference in their entirety.

This structure has been modified by synthetic procedures, where uranium and titanium cations were isomorphically replaced by metals with variable oxidation states, chiefly by vanadium and molybdenum cations. These procedures are reported in the following scientific articles: Machej T., Kozlowski R. and Ziolkowski J. J. of Solid State Chem. 38, (1981) 97; Ziolkowski J., Krupa K. and Mocala K. J. of Solid State Chem. 48, (1983) 376; Mocala K., Ziolkowski J. and Dziembaj L. J. of Solid State Chem. 56 (1985) 84; Mocala K. and Ziolkowski J. J. of Solid State Chem. 71 (1987) 426; Mocala K. and Ziolkowski J. J. of Solid State Chem. 71 (1987) 552; Maslowska B. and Ziolkowski J. J. of Solid State Chem. 87 (1990) 208. The polymorphism of the bivalent metal vanadates MeV₂O₆ (Me=Mg, Ca, Mn, Co, Ni, Cu, Zn and Cd) has been also studied by Mocala K. and Ziolkowski J. J. of Solid State Chem. 69 (1987) 299.

The application of MnVMoO brannerite-like structure compounds was formerly intended for partial oxidation of propylene; Ziólkowski J. and Janas J. J. Catal., 81, 2, (1983) 298; Ziólkowski J. J. Catal., 81, 2, (1983) 311 and in the partial oxidation of o-xylene; Ziolkowski J. and Gasior M. J. Catal., 84 (1983) 74. In all cases, the oxidation reactions were used as molecules test to assess the reaction mechanisms and the role of the crystalline planes.

However, the catalytic performance obtained by the formulations reported so far, was not sufficient to envisage an industrial application of these materials, thus, no further research on this application was conducted.

On the other hand, the brannerite-like compounds have been also widely studied and used as cathodes in rechargeable lithium batteries, as was reported in the later 1990s by J. B. Gooeneugh et al, Denki Kagaku, 66 (1173) 1998; and S. R. Prabaharan, M. S. Michael, Abstracts of Mater. Res. Soc. Symp. Fall meeting 1998 (Boston); number EE3.26. In “Synthesis, structure and lithium intercalation reaction in LiMoVO₆ brannerite-type materials”. J. Mater. Chem., 2003, 13, 2374-2380, C. M., Julien et. al. describe a LiMoVO₆ with brannerite-type structure synthesized by various methods. This material is used for the manufacture of positive electrodes for rechargeable batteries.

US 2003/0235761A1 to Prabaharan describes a method to prepare the layered compound LiVMoO_{5.5} by the use of aqueous solutions. The material can be prepared by sol-gel methods, co-precipitation, hydrothermal, soft combustion processes and processes involving complexation agents. Lithium, vanadium and molybdenum oxide, with brannerite-like structure are disclosed as being used as a cathode in rechargeable lithium containing batteries.

R. S. Liu et al, in “A Novel Anode Material LiVMoO₆ for Rechargeable Lithium-Ion Batteries; Electroch. & Sol. Stat. Lett., 8 (12) A650-A653 (2005), report the preparation of LiVMoO₆ multi-component oxide by the solid state reaction method, for its use as an anode in lithium rechargeable batteries.

Normally, this type of LMMO has been synthesized by several known procedures summarized as follows: a) Solution of the precursors of different constituents, drying to recover a solid and subsequent thermal treatment; b) Solution of the precursors of different constituents, hydrothermal treatment to recover a solid and subsequent thermal treatment; c) Solid mixture of different constituents and subsequent thermal treatments, d) Solid mixture of the precursors of different constituents, impregnation with organic chemical agents, and subsequent thermal or hydrothermal treatments.

Among these methods, particular attention deserves the solid state reaction synthesis, which involves repeated steps of mixing and heating during long periods of time, in order to complete the whole reaction. This method has been extensively employed for producing these types of compounds. However, it is difficult to control of the homogeneity and size of the particles by this method, because of the reaction between the reactants is through the grain's frontiers.

According to the state of the art, these LMMO or its derivatives have not been used as catalysts for the oxidative dehydrogenation of ethane for producing ethylene.

### SUMMARY OF THE INVENTION

The previous technologies known by the applicant were overcome by the present invention, as none of the cited references relates to layered multimetallic mixed oxides (LMMO) for use in the petrochemical industry as catalysts in the oxidative dehydrogenation of ethane for producing ethylene.

To overcome the problems of the prior processes, the use of homogeneous media is proposed, particularly; the use of solution wherein the reactants are intimately related, hence the interaction is performed at atomic or molecular level, which allows a better control of the physicochemical properties, and where the synthesis does not need long periods of time nor high temperatures to be completed.

Therefore, one of the objects of the present invention is to provide a catalyst and to a process for preparing catalysts based on LMMO, with catalytic properties for the oxidative dehydrogenation of ethane for producing ethylene.

Another object of the present invention is to provide LMMO as catalysts for the oxidative dehydrogenation of ethane to produce ethylene, which is represented by the general formula:

M1 M2 M3 O_{δ}

wherein
M1 is selected from the group of Ag, Au, Zn, Sn, Rh, Pd, Pt, Cu, Ni, Fe, Co, an alkaline metal, an alkaline earth metal, a rare earth, and mixtures thereof.
M2 is selected from the group of Ti, Hf, Zr, Sn, Bi, Sb, V, Nb, Ta and P, and mixtures thereof.
M3 is selected from the group of Mo, W and Cr, and mixtures thereof.
δ depends on the amount and oxidation state or valence of the components, the starting materials, preparation method and the activation process
and where the catalyst exhibits at least one X-ray diffraction peak between 5<2θ<15.

An additional object of the present invention is to provide an activation process of the LMMO. This process can be important for obtaining an active and selective catalyst for the oxidative dehydrogenation of ethane for producing ethylene.

An additional object of the present invention is to provide a LMMO catalyst for the partial oxidation of light hydrocarbons, and particularly for the oxidative dehydrogenation of the oxidative dehydrogenation of ethane for producing ethylene.

The catalyst according to one or embodiment of the invention is a multilayered mixed metallic oxide that exhibits at least one X-ray diffraction peak between 5<2θ<15, and preferably 10<2θ<15.

Another object of the invention is to provide a mixed multimetallic metal oxide catalyst that can exhibits X-ray diffraction peaks of monoclinic lattice of silver vanadium molybdenum oxide corresponding to ICDD-PDF 04-002-4830, or cesium vanadium molybdenum oxide corresponding to ICDD-PDF 00-030-0381, or monoclinic sodium vanadium molybdenum oxide corresponding to ICDD-PDF 04-011-9693, or monoclinic lithium vanadium molybdenum oxide corresponding to ICDD-PDF 04-006-7234, or orthorhombic calcium vanadium molybdenum oxide corresponding to ICDD-PDF 04-013-4035.

These and other features of the invention will become apparent from the following description of the invention which discloses various embodiments of the invention.

### BRIEF DESCRIPTION OF THE DRAWINGS

FIG. 1 is an X-ray pattern of LiNb_{0.5}V_{0.5}Mo of Example 1 that was calcined in an air static atmosphere at 550° C. for 2 hours.

FIG. 2 is an X-ray pattern of LiVMo of Example 2 that was calcined in an air static atmosphere at 550° C. for 24 hours.

FIG. 3 is an X-ray pattern of Li_{0.8}Zn_{0.2}V_{1.5}Mo_{0.8} of Example 5 that was calcined in an air static atmosphere at 550° C. for 24 hours.

FIG. 4 is an X-ray pattern of Li_{0.8}Ag_{0.2}VMo of Example 9 that was calcined in an air static atmosphere at 550° C. for 24 hours.

FIG. 5 is a High Resolution of Transmission Electronic Microscopy image of the Li_{0.8}Ag_{0.2}VMo catalyst, prepared according to Example 9 where (A) is an image of a catalyst's particles and (B) is a close-up image of a particle.

FIG. 6 is an X-ray pattern of LiVMo of Example 14 that was calcined in an air static atmosphere at 550° C. for 4 hours.

FIG. 7 is an X-ray pattern of LiVMo of Example 15 that was calcined in an air static atmosphere at 550° C. for 4 hours.

FIG. 8 is an X-ray pattern of LiVMo of Example 16 that was calcined in an air static atmosphere at 550° C. for 4 hours.

FIG. 9 is an X-ray patterns of LiVMo of Example 18 that was calcined in an air static atmosphere at 550° C. for 4 hours.

FIG. 10 is an X-ray pattern of material with atomic composition LiVMo of Examples 19 that was calcined in an air static atmosphere at 550° C. during 4 hours.

FIG. 11 is an X-ray pattern of LiVMo of Example 20 that was calcined in an air static atmosphere at 550° C. for 4 hours.

FIG. 12 is an X-ray pattern of Li_{1.2}V_{1.2}Mo_{0.88} of Example 21 that was calcined in an air static atmosphere at 550° C. for 4 hours.

FIG. 13 is an X-ray pattern of NaVMo of Example 22 that was calcined in an air static atmosphere at 550° C. for 4 hours.

FIG. 14 is an X-ray pattern of AgVMo of Example 23 that was calcined in an air static atmosphere at 550° C. for 4 hours.

FIG. 15 is an X-ray pattern of Li_{0.8}Zn_{0.2}V_{1.5}MO_{0.8} of Example 24 that was calcined in an air static atmosphere at 550° C. for 4 hours.

FIG. 16 is an X-ray pattern of Li_{0.8}Ag_{0.2}VMo of Example 25 that was calcined in an air static atmosphere at 550° C. for 4 hours.

FIG. 17 is an X-ray pattern of material with formula AgVMo of Example 26 that was calcined in an air static atmosphere at 550° C. for 4 hours.

FIG. 18 is a High Resolution of Transmission Electronic Microscopy image of the catalyst prepared in Example 26 where (A) is an image of catalyst's crystal with the following atomic composition: AgVMo. The particle is decorated by nanometric Ag metallic particles highly dispersed (B) close-up of the metallic particles, which were indexed as metallic silver. The catalyst was thermally treated at 550° C. under air atmosphere for 24 hours.

### DETAILED DESCRIPTION OF THE INVENTION

The present invention is directed to a catalyst and to a process for preparing the catalysts based on layered multimetallic mixed oxides (LMMO), which exhibit a layered structure characterized by at least one X-ray diffraction peak or a plurality of peaks located between 5<2θ<15, and preferably between 10<2θ<15. The presence of additional diffraction peaks may be observed, indicating the existence of other crystalline phases. Crystalline arrangements have been identified as monoclinic lattice of Silver Vanadium Molybdenum Oxide (corresponding to ICDD-PDF 04-002-4830), Cesium Vanadium Molybdenum Oxide (corresponding to ICDD-PDF 00-030-0381), monoclinic Sodium Vanadium Molybdenum Oxide (corresponding to ICDD-PDF 04-011-9693), monoclinic Lithium Vanadium Molybdenum Oxide (corresponding to ICDD-PDF 04-006-7234), and orthorhombic Calcium Vanadium Molybdenum Oxide (corresponding to ICDD-PDF 04-013-4035), among other crystalline structures. This invention also relates to a process for the oxidation or partial oxidation of light hydrocarbons using the LMMO catalysts. The invention is particularly directed to a process for producing ethylene and catalysts for the oxidative dehydrogenation of low molecular weight hydrocarbons, preferably between 2 and 4 carbon atoms. More particularly the invention refers to a process for the oxidative dehydrogenation of ethane to ethylene by contacting a stream of ethane with the catalysts of the present invention.

The LMMO catalysts of the present invention can be represented by the general formula:

M1 M2 M3 O_{δ}

wherein
M1 is selected from the group of Ag, Au, Zn, Sn, Rh, Pd, Pt, Cu, Ni, Fe, Co, an alkali metal, an alkaline earth metal, a rare earth, and mixtures thereof.
M2 is selected from the group of Ti, Hf, Zr, Sn, Bi, Sb, V, Nb, Ta and P, and mixtures thereof.
M3 is selected from the group of Mo, W and Cr, and mixtures thereof.
δ depends on the amount and oxidation state or valence of the components, the starting materials, preparation method and the activation process.

In one embodiment of the invention, the catalyst is represented by the formula

M1ₓ M2_{y} M3_{z} O_{δ}

where M1, M2, M3 and 6 are as defined above, and where x, y and z are >0 and are dependent on the valence and oxidation state of the metals. Examples of catalysts for the production of ethylene from ethane include oxides of LiNbVMo, LiVMo, LiZnVMo, Li_{0.8}Zn_{0.2}V_{1.2}Mo_{0.8}, Li_{0.8}Ag_{0.2}VMo, Li_{1.2}V_{1.2}Mo_{0.8}, and AgVMo. In one embodiment of the invention, M1 is an alkali metal such as Li, M2 is V and M3 is Mo. In another embodiment, M1 is a mixture of Li an Ag.

The mixed metallic oxide catalyst of the invention exhibit various 2θ peak positions of the X-ray diffraction pattern that are substantially the same as or reasonably resemble the peaks found in a ICDD-PDF card for certain compounds. The powder diffraction files (PDF) are available from the International Center for Diffraction Data (ICDD).

In one embodiment of the invention, the layered multimetallic mixed oxide includes a brannerite-type crystal structure in combination with other crystalline phases. In other embodiments, the layered multimetallic mixed oxide can have a brannerite-type crystal structure as the primary crystal phase. The crystalline structure will vary depending on the chemical composition of the oxides. For example, Li has been found to produce a brannerite structure. Ag has been found to produce a crystalline structure that is different from a brannerite phase or structure. A combination of Li and Ag produce a mixture of crystalline phases with significant X-ray diffraction peaks at 2θ ranging from 10 to 15. As shown in the Examples, AgVMo oxides exhibit crystalline phase that are identified in the ICDD-PDF library.

The process for producing the mixed multimetallic oxides comprises the steps of

mixing, either in solid or liquid phase, the metal precursor compounds where the pH of the resulting liquid mixture phase optionally can be adjusted;

thermal or hydrothermal treatment of the resulting mixture obtained in the first step to obtain a solid component;

drying the resulting solid component obtained in the second stage; and

thermally treating the resulting dried solid obtained in the third stage to obtain the activated solid catalyst.

A further embodiment of the invention is directed to a process for the oxidative dehydrogenation of ethane to ethylene, where ethane and an oxygen containing gas are contacted in the presence of a multimetallic oxide catalyst under conditions to oxidatively dehydrogenate ethane to produce ethylene, wherein the catalyst is represented by the general formula:

M1 M2 M3 O_{δ}

where M1, M2, M3 and O₆ are as defined above.

### Preparation of the Layered Multimetallic Mixed Oxides (LMMO):

The present invention provides a process for the preparation of nanometric and micrometric LMMO, comprising the steps of:
i) preparing a mixture of metallic precursors either by mechanical mixing of the powder or dissolution of the metallic salt precursors, where the pH of the mixture optionally can be adjusted.
ii) the optional addition of an organic chemical agents to the mixture of step i),
iii) When the mixture is obtained by mechanical mixing of the powders, the mixture optionally can be dissolved in water.
iv) Perform a hydrothermal treatment of the resulting mixture of the metallic precursors.
v) Drying of the hydrothermally treated mixture of step iv) and recovering a homogeneous solid,
vi) Activation of the solid obtained in step v) by thermal treatments to obtain a catalyst for the oxidative dehydrogenation of ethane to ethylene.

The precursors of the catalyst are selected from the group of Ag, Au, Zn, Sn, Rh, Pd, Pt, Cu, Ni, Fe, Co, Ti, Hf, Zr, Sn, Bi, Sb, V, Nb, Ta, P, Mo, W, Cr, rare earth metal, alkaline earth metal and alkaline metal, and mixtures thereof. The precursors can be incorporated to the mixing stage as pure metallic elements, metallic salts, metallic oxides, metallic hydroxides, metallic alkoxides, acids or as a mixture thereof. Preferably, the precursors are incorporated as nitrates, oxalates, sulfates, carbonates or halides more preferably nitrate salts. In a preferred embodiment, the mixture of precursors is obtained by mixing a metal or metal salt of M1, M2 and M3 where M1, M2 and M3 are as defined above.

The pH of solution of the precursors of the elements constituting the catalyst, in step i) can be adjusted with mineral acids, or organic or inorganic bases, such as H₂SO₄, HNO₃, HCl, NH₄OH or mixtures thereof.

The addition of organic chemical agents to the solution of step i) can be before or after pH adjustment.

The evaporation of the solvent in step v) can be performed by conventional methods, such as evaporation in an oven, evaporation in vacuum, spray-drying, or by a combination thereof.

The solid of the LMMO precursor's mixture, prepared in step v) is dried at a temperature ranging between 80 and 120° C. The drying step can be in an oxidizing, reducing or inert atmosphere for 1 to 5 hours at a heating rate of 0.1 to 5° C./minute. The oxidizing atmosphere is selected from the group consisting of oxygen, air, carbon dioxide, ozone, and mixtures thereof. The reducing atmosphere is selected from the group consisting of hydrogen, CO, lower alcohols, H₂O₂, light hydrocarbons, and mixtures thereof. The inert atmosphere is selected from the group consisting of nitrogen, argon, helium, and mixtures thereof.

The dried solids obtained in step v), are subjected to activation by thermal treatment under an oxidizing atmosphere or an oxidizing or reducing atmosphere flow, at temperatures ranging between 400 and 900° C., more preferably from 550 to 700° C., for a period of time ranging between 1 and 48 hours, preferably between 2 and 48 hours, and a heating rate between 1 and 5° C./min.

The LMMO can also be supported over a solid, such as silica, silica-gel, amorphous silica, zirconium oxide, alumina, titanium oxide, aluminum-silicates or mixtures thereof. Preferably, the solid support is present in an amount of 20 to 70% weight on the total weight of the catalyst composition. When the selected support is silica, the preferred sources of silicon are colloidal silica and/or amorphous silica. The impregnation of the different chemical components of the catalyst over the support can be performed by conventional methods of impregnation such as by applying an excess solution, incipient wetness or by precipitation over the support of an aged solution or a fresh solution containing the precursor of the active chemical elements.

In one embodiment of the preparation of LMMO, the metallic precursors or salts thereof are selected from the group of, Ag, Au, Zn, Sn, Rh, Pd, Pt, Cu, Ni, Fe, Co, Ti, Hf, Zr, Sn, Bi, Sb, V, Nb, Ta, P, Mo, W and Cr, rare earth metal, alkaline earth metal and alkaline metal and are mixed in the mixing step by mechanical grinding of the solid precursors. In the process, a low molecular weight alcohol or acetone can be used as a homogenizing agent of the solid mixture, which leads to the formation of a homogeneous paste.

In another process for the preparation of LMMO, the mixing step of the metallic precursors or salts thereof selected from the group of Ag, Au, Zn, Sn, Rh, Pd, Pt, Cu, Ni, Fe, Co, Ti, Hf, Zr, Sn, Bi, Sb, V, Nb, Ta, P, Mo, W and Cr, rare earth metal, alkaline earth metal and alkaline metal, is performed by the dissolution of solid precursors. The mixture obtained can be subjected to a period of static permanence or in agitation within the reactor. Then, the resulting solution is heated at temperatures between 50 and 100° C., and later is subjected to an evaporation process to remove the solvent.

In another process for the preparation of LMMO, the mixing stage of the metallic precursor or salts thereof selected from the group of Ag, Au, Zn, Sn, Rh, Pd, Pt, Cu, Ni, Fe, Co, Ti, Hf, Zr, Sn, Bi, Sb, V, Nb, Ta, P, Mo, W and Cr, rare earth metal, alkaline earth metal or alkaline metal, is performed by the dissolution of solid precursors. The resulting mixture can be subjected to a period of static permanence or in agitation within the reactor. Then, the resulting solution is subjected to a hydrothermal treatment, where the temperature and time of treatment are performed between 50 and 250° C., preferably between 50 and 150° C., for 2 and 200 hours, preferably from 4 to 50 hours.

In another process for the preparation of the LMMO, the mixing stage of the metallic precursors or salts thereof selected from the group of Ag, Au, Zn, Sn, Rh, Pd, Pt, Cu, Ni, Fe, Co, Ti, Hf, Zr, Sn, Bi, Sb, V, Nb, Ta, P, Mo, W and Cr, rare earth metal, alkaline earth metal or alkaline metal, is performed by the dissolution of solid precursors. Then, an aqueous solution of an organic compound selected from an amino acid, preferably glycine, amines, urea or carboxylic acids, or a mixture thereof, is added to the solution of solid precursors. The pH of the final solution can be lightly acid or neutral, preferably neutral, adjusting the pH by the use of a base or acid, as required. Then, the solution is subjected to an evaporation process for removing the solvent. Finally, the resulting solid is subjected to a slow heating process at temperatures ranging from 300 to 400° C., with a heating rate between 0.5 and 1.5° C./min, during a time between 0.5 and 2 hours.

In another process for the preparation of LMMO, a reducing agent is introduced to the synthesis method. The mixing stage of the metallic precursors or salts thereof selected from the group of Ag, Au, Zn, Sn, Rh, Pd, Pt, Cu, Ni, Fe, Co, Ti, Hf, Zr, Sn, Bi, Sb, V, Nb, Ta, P, Mo, W and Cr, rare earth metal, alkaline earth metal or alkaline metal, is performed by mechanical grinding of the solid precursors. The resulting solid mixture is then impregnated, by incipient wetness method, with an aqueous solution of an organic reducing agent, preferably selected from the group of: hydrazine, oxalate, amines or urea, or a mixture thereof. The concentration of organic reducing agent ranges between 0.1 and 1.5 moles per mol of multimetallic mixed oxide, preferably between 0.2 and 1.5 moles per mol of multimetallic mixed oxide and more preferably between 0.2 and 0.9 moles per mol of multimetallic mixed oxide. Once impregnated the mixture of the salts with the organic reducing agent, the LMMO can be obtained by followings treatments:
1. The slurry is maintained at room temperature for 1 to 8 hours, preferably between 2 and 4 hours. Then, it is dried at 80-120° C. for 4 and 12 hours.
2. The slurry is autoclaved to start a hydrothermal treatment at a temperature ranging from 40 to 250° C., preferably between 80 and 150° C., for 1 to 48 hours, preferably between 2 and 12 hours. The solid recovered from the hydrothermal treatment is dried at a temperature between 80 and 150° C. for 4 to 12 hours.
3. The slurry is dissolved in distilled water and later the solution is subjected to hydrothermal treatment. The solution is hydrothermally treated and placed in a rotavapor to be subjected to a solid recovery process by water evaporation. The recovered solid is dried at 80-150° C. for a period between 4 and 12 hours.

The layered multimetallic mixed oxide precursor mixture, prepared by any of the above describe procedures, is then subject to the same drying, and/or hydrothermal and/or thermal treatments to obtain the activated LMMO, which is used as catalyst in the oxidative dehydrogenation of ethane to ethylene.

### Activation Process of Multi-Metallic Oxide

The activation procedure of the LMMO includes the thermal treatment of the dried solids, obtained in the fourth step. The thermal treatment can be performed under an oxidant atmosphere and/or an oxidant or reducing flow, and/or inert flow, at temperatures ranging between 400 and 900° C., more preferably from 550 to 700° C., for a period of time ranging from 2 to 48 hours and heating rate between 1 and 5° C./min.

The oxidant agent of the thermal treatment can be oxygen, air, carbon dioxide, ozone or mixtures thereof, preferably oxygen, and more preferably air.

The inert gas of the thermal treatment can be nitrogen, argon, helium or mixture thereof.

The reducing agent of the thermal treatment can be hydrogen, CO, alcohols, H₂O₂, light hydrocarbons as methane, or a mixture of two or more reducing agents.

Once the solid has been thermally activated, the catalysts prepared according to any of the above described procedures, is in a suitable form to perform the oxidative dehydrogenation of ethane for producing ethylene.

### Application of the Activated LMMO in the ODH-et for Producing Ethylene

The oxidative dehydrogenation of ethane (ODH-Et) to ethylene is performed by contacting ethane with an oxidizing agent and/or an inert agent using an activated LMMO catalyst.

In the conversion of ethane to ethylene, the oxidizing agent can be oxygen, air, carbon dioxide, ozone or a mixture thereof, preferably oxygen, and more preferably air.

In the conversion of ethane to ethylene, the inert agent can be nitrogen, argon, helium or a mixture thereof.

In the conversion of ethane to ethylene, the ODH-Et in gaseous phase is performed in the presence of water vapor. The content of water can vary from 5 to 80% by mole, preferably from 20 to 60% by mole.

In the conversion of ethane to ethylene, the ODH-Et in the gaseous phase is performed using as catalyst an activated LMMO loaded in a fixed or fluidized bed reactor.

The conversion of ethane to ethylene can be carried out in a fixed bed reactor. The catalyst is diluted with an inert material for helping to dissipate heat generated in the catalytic bed and avoiding the hot point formation.

In the conversion of ethane to ethylene, silicon carbide or alpha-alumina or other ceramic material is used as a diluent of the catalytic bed.

In the conversion of ethane to ethylene, the ODH-Et is performed in a fixed bed reactor at a reaction temperature between 300 and 700° C., preferably between 400 and 600° C., an more preferably between 500 and 600° C.

The conversion of ethane and ethylene is performed in a fixed bed reactor, a contact time, W/F°ₑₜₕₐₙₑ, defined as the ratio between the catalyst mass and the molar flow of ethane fed to the reactor, ranges from 0.01 and 50.0 grams of catalyst hour per mol of ethane fed (g_{cat} h/molₑₜₕₐₙₑ), preferably between 0.05 and 25.0 g_{cat} h/molₑₜₕₐₙₑ, more preferably between 0.1 and 15.0 g_{cat} h/molₑₜₕₐₙₑ.

The catalytic conversion of ethane to ethylene is typically performed by a flow of ethane, an oxidant agent and a carrier gas over a bed of the catalyst at a temperature, pressure and flow rate to produce ethylene. In one embodiment, the reaction mixture contains a molar content of ethane of at least 30 mole %. The ethane content of the reaction mixture can range from about 20-40 mole % with the balance oxygen, nitrogen, and mixtures thereof. One example of a suitable reaction mixture is a mixture of ethane, oxygen and nitrogen in a molar ratio of 30/10/60. The process of the invention is able to produce ethylene in an amount of equal to or greater than 1800 grams per hour and per Kg of catalyst at atmospheric pressure. In another embodiment, ethylene is produced at a rate of at least 600 grams per hour per Kg of catalyst at atmospheric pressure.

### EXAMPLES

The following examples are illustrative of some of the products and methods of making and using the catalyst, falling within the scope of the present invention. They are not to be considered in any way limiting of the invention.

In the Examples, reference is made to room pressure and temperature, and refers to the following values:

Room temperature=10 to 35° C., room pressure=500 to 760 mmHg.

### Example 1

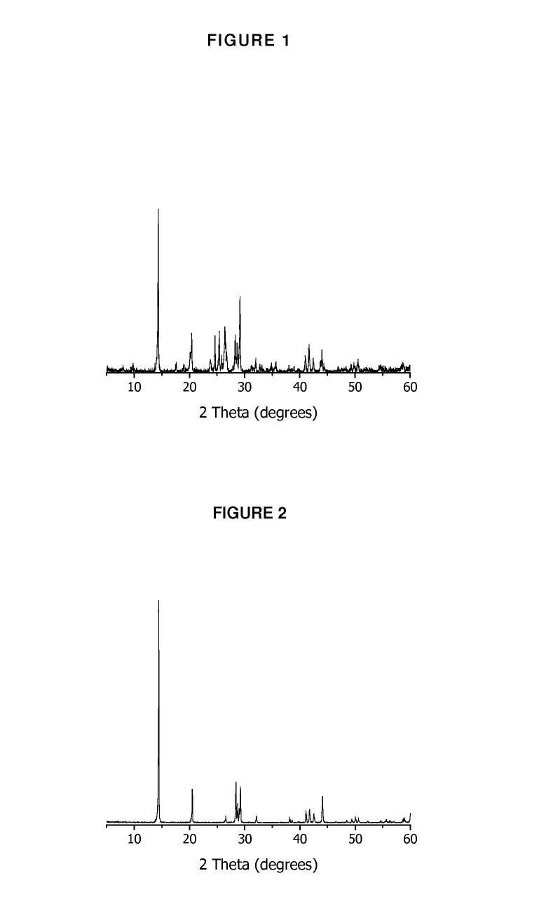

Preparation of a LMMO of LiNbVMo oxide. The preparation of the LMMO was performed by the solid state reaction method for obtaining a catalyst with atomic composition: LiNb_{0.5}V_{0.5}Mo. For that, 2.551 grams of lithium nitrate (LiNO₃), 1.080 grams of ammonium metavanadate (NH₄VO₃), 6.519 grams of ammonium heptamolybdate ((NH₄)₆Mo₇O₂₄4.H₂O) and 8.030 grams of niobium oxalate (C₁₀H₈N₂Nb₂) were mixed for 1 hour in an agate mortar, adding acetone to facilitate and improve the mixing and the homogeneity of the components, until obtaining a yellow solid paste. Then, the paste is dried in an oven at 110° C. for 1 hour under air static atmosphere, and then ground again to obtain a yellow powder. The dried yellow powder was thermally treated in a muffle under air static atmosphere, at 300° C. for 1 hour and at 550° C. for 2 hours. The X-ray diffraction pattern of this material is shown in FIG. 1. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Other crystalline phases were also detected with diffraction peaks located at 2θ: 7.98, 9.80, 17.60, 19.04, 20.14, 23.82, 24.64, 25.42, 25.86, 31.24, 32.76, 34.88 and 35.72.

The catalytic results of the ODH-Et obtained with the LiNb_{0.5}V_{0.5}MoO catalyst are shown in Table 1. The reaction was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 600° C. for 1 hour.

### Example 2

Preparation of a LMMO of LiVMo oxide. The preparation of the LMMO was performed by the solid state reaction method for obtaining a catalyst with atomic composition: LiVMo. For that, 2.27 grams of lithium nitrate (LiNO₃), 3.86 grams of ammonium metavanadate (NH₄VO₃) and 5.83 grams of ammonium heptamolybdate ((NH₄)₆Mo₇O₂₄.4H₂O) are mixed for 1 hour in an agate mortar, adding ethanol to facilitate and improve the mixing and the homogeneity of the components to obtain a yellow solid paste. Then, the paste is dried in an oven at 110° C. for 1 hour under air static atmosphere, and then ground again in an agate mortar to obtain a yellow powder. The dried yellow powder was thermally treated in a muffle under air static atmosphere, at 300° C. for 1 hour and 550° C. for 24 hours. The X-ray pattern of this catalyst is shown in FIG. 2. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17.

The main catalytic results of the ODH-Et obtained with the LiVMo catalyst are shown in Table 1. The reaction was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 600° C. for 1 hour.

### Example 3

The ODH-Et reaction performed over the LiVMo oxide catalyst of Example 2, was performed also at 630° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 630° C. for 1 hour. The catalytic results of the ODH-Et obtained with the LiVMo catalyst are shown in Table 1.

### Example 4

The ODH-Et reaction with the catalyst LiVMo oxide of Example 2 was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 630° C. for 1 hour. The main catalytic results of the ODH-Et obtained with the LiVMo catalyst are shown in Table 1.

### Example 5

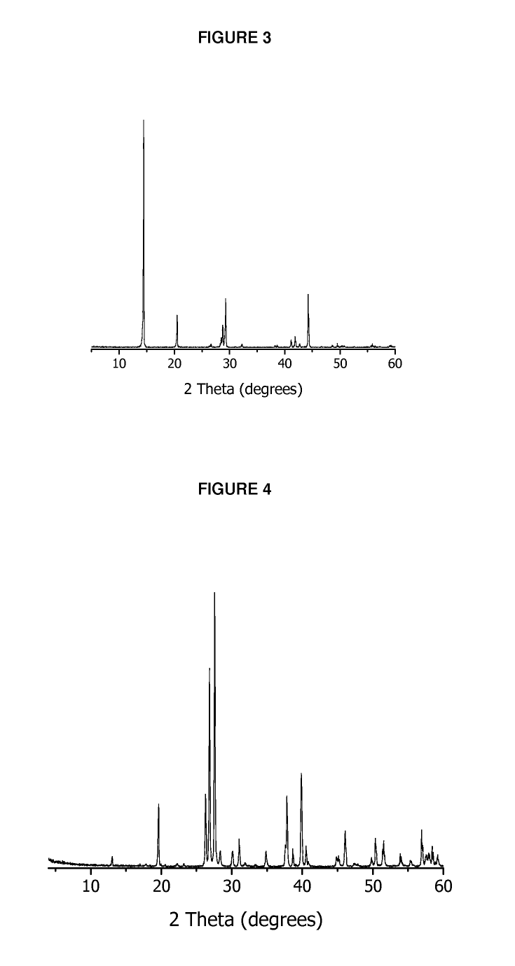

Preparation of a LMMO of LiZnVMo oxide. The preparation of the LMMO was performed by the solid state reaction method for obtaining a catalyst with the following atomic composition: Li_{0.8}Zn_{0.2}V_{1.2}Mo_{0.8} oxide. For that, 2.7570 grams of lithium nitrate (LiNO₃), 2.4435 grams of zinc nitrate (Zn(NO₃)₂.6H₂O), 7.02 grams of ammonium metavanadate (NH₄VO₃) and 7.062 grams of ammonium heptamolybdate ((NH₄)₆Mo₇O₂₄4.H₂O) are mixed for 1 hour in an agate mortar, adding ethanol to facilitate and improve the mixing and the homogeneity of the components to obtain a yellow solid paste. Then, the paste was dried in an oven at 110° C. for 1 hour under air static atmosphere, and ground again in an agate mortar to obtain a yellow powder. The dried yellow powder is thermally treated in a muffle under air static atmosphere, at 300° C. for 1 hour and 550° C. for 24 hours. The X-ray pattern of this catalyst is shown in FIG. 3. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Other crystalline phases were also detected with diffraction peaks located at 2θ: 41.88, 44.26 and 44.38.

The main catalytic results of the ODH-Et obtained with the Li_{0.8}Zn_{0.2}V_{1.2}MO_{0.8} catalyst are shown in Table 1. The reaction was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 630° C. for 1 hour.

### Example 6

The ODH-Et reaction with the Li_{0.8}Zn_{0.2}V_{1.2}Mo_{0.8} oxide catalyst of Example 5 was performed at 630° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 630° C. for 1 hour. The main catalytic results of the ODH-Et obtained with the Li_{0.8}Zn_{0.2}V_{1.2}Mo_{0.8} catalyst are shown in Table 1.

### Example 7

The ODH-Et reaction with the Li_{0.8}Zn_{0.2}V_{1.2}Mo_{0.8} oxide catalyst of Example 5 was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min helium at 600° C. for 1 hour. The catalytic results of the ODH-Et obtained with the Li_{0.8}Zn_{0.2}V_{1.2}Mo_{0.8} oxide catalyst are shown in Table 1.

### Example 8

The ODH-Et reaction with the Li_{0.8}Zn_{0.2}V_{1.2}Mo_{0.8} oxide catalyst of Example 5 was performed at 630° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 600° C. for 1 hour. The main catalytic results of the ODH-Et obtained with the Li_{0.8}Zn_{0.2}V_{1.2}Mo_{0.8} oxide catalyst are shown in Table 1.

### Example 9

Preparation of a LMMO of LiAgVMo. oxide The preparation of the LMMO was performed by the solid state reaction method for obtaining a catalyst with the following atomic composition: Li_{0.8}Ag_{0.2}VMo oxide. 2.7570 grams of lithium nitrate (LiNO₃), 1.70 grams of silver nitrate (AgNO₃), 5.8475 grams of ammonium metavanadate (NH₄VO₃) and 8.8276 grams of ammonium heptamolybdate ((NH₄)₆MO₇O₂₄.4H₂O) were mixed for 1 hour in an agate mortar, adding acetone to facilitate and improve the mixing and the homogeneity of the components to obtain a yellow solid paste. Then, the paste is dried in an oven at 110° C. for 1 hour on air static atmosphere, and ground again to obtain a yellow powder. The dried yellow powder is thermally treated in a muffle under air static atmosphere, at 300° C. for 1 hour and 550° C. for 24 hours. The X-ray pattern of this catalyst is shown in FIG. 4. XRD pattern of LMMO can be indexed to silver vanadium molybdenum mixed oxide (ICDD-PDF 04-002-4830) with main diffraction peaks located at 2θ: 19.56, 26.35, 26.77, 27.58, 31.09, 34.83, 37.58, 44.88, 49.88, and 51.54. Other crystalline phases were also detected with diffraction peaks located at 2θ: 30.14, 40.54, and 56.92. A small amount of LiVMo was also detected.

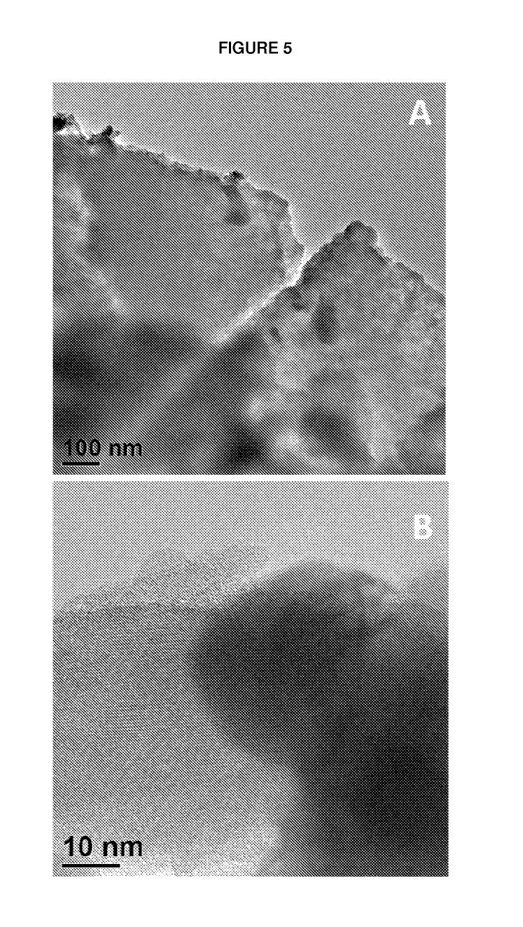

On this sample a high resolution of transmission electronic microscopy (HRTEM) study was performed. FIG. 5 is representative of the LiAgVMo oxide catalyst. (A) Image of representative catalyst's particles (B) close-up of a particle. The FIG. 5 (A) clearly shows a layered arrangement of the material, which confirm those observed by XRD. The EDS results show that the material is chemically uniform.

The main catalytic results of the ODH-Et obtained with the Li_{0.8}Ag_{0.2}VMo oxide catalyst are shown in Table 1. The reaction was performed at 500° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 550° C. for 1 hour.

### Example 10

The ODH-Et reaction with the Li_{0.8}Ag_{0.2}VMo oxide catalyst of Example 9 was performed at 550° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 550° C. for 1 hour. The main catalytic results of the ODH-Et obtained with the Li_{0.8}Ag_{0.2}VMoO₆ oxide catalyst are shown in Table 1.

### Example 11

The DHO-Et reaction with the Li_{0.8}Ag_{0.2}VMo oxide catalyst of Example 9 was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 600° C. for 1 hour. The main catalytic results of the ODH-Et obtained with the Li_{0.8}Ag_{0.2}VMo oxide catalyst are shown in Table 1.

### Example 12

The ODH-Et reaction with the Li_{0.8}Ag_{0.2}VMo oxide catalyst of Example 9 was performed at 630° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 600° C. for 1 hour. The main catalytic results of the ODH-Et obtained with the Li_{0.8}Ag_{0.2}VMo oxide catalyst are shown in Table 1.

### Example 13

The ODH-Et reaction with the Li_{0.8}Ag_{0.2}VMo oxide catalyst of Example 9 was performed at 600° C. using an ethane/oxygen/nitrogen flow with molar ratio of 30/10/60 and a W/F°ₑₜₕₐₙₑ=7.1 g_{cat} h/molₑₜₕₐₙₑ. Prior to the catalytic activity measurement, the catalyst was pretreated with a flow of 100 ml/min of helium at 630° C. for 1 hour. The main catalytic results of the ODH-Et obtained with the Li_{0.8}Ag_{0.2}VMo oxide catalyst are shown in Table 1.

TABLE 1
Main catalytic results of the ODH-Et for producing ethylene,
using the activated LMMO of the present invention, examples 1 to 13.

| | | Reaction | Ethylene | |
|---|---|---|---|---|
| | | temperature, | selectivity, | Ethylene |
| Example | Catalyst | ° C. | % mol | production^{a} |
| 1 | LiNb_{0.5}V_{0.5}Mo | 600 | 66 | 1,041 |
| 2 | LiVMo | 600 | 76 | 569 |
| 3 | LiVMo | 630 | 63 | 1,292 |
| 4 | LiVMo | 600 | 72 | 937 |
| 5 | Li_{0.8}Zn_{0.2}V_{1.2}Mo_{0.8} | 600 | 70 | 1,877 |
| 6 | Li_{0.8}Zn_{0.2}V_{1.2}Mo_{0.8} | 550 | 68 | 1,502 |
| 7 | Li_{0.8}Zn_{0.2}V_{1.2}Mo_{0.8} | 600 | 82 | 808 |
| 8 | Li_{0.8}Zn_{0.2}V_{1.2}Mo_{0.8} | 630 | 63 | 1,168 |
| 9 | Li_{0.8}Ag_{0.2}VMo | 500 | 68 | 1,314 |
| 10 | Li_{0.8}Ag_{0.2}VMo | 550 | 72 | 1,760 |
| 11 | Li_{0.8}Ag_{0.2}VMo | 600 | 69 | 844 |
| 12 | Li_{0.8}Ag_{0.2}VMo | 630 | 61 | 1,058 |
| 13 | Li_{0.8}Ag_{0.2}VMo | 600 | 73 | 1,036 |

^{a}Units: grams of ethylene produced per hour per kilogram of catalyst

### Example 14

Preparation of a LMMO of LiVMo. The preparation of the LMMO was through the following steps: 1) precursors salts are dissolved, 2) the solution is hydrothermally treated, and 3) The solvent, of the hydrothermally treated precursors salts solution, is evaporated to obtain a catalyst with the following atomic composition: LiVMo oxide.

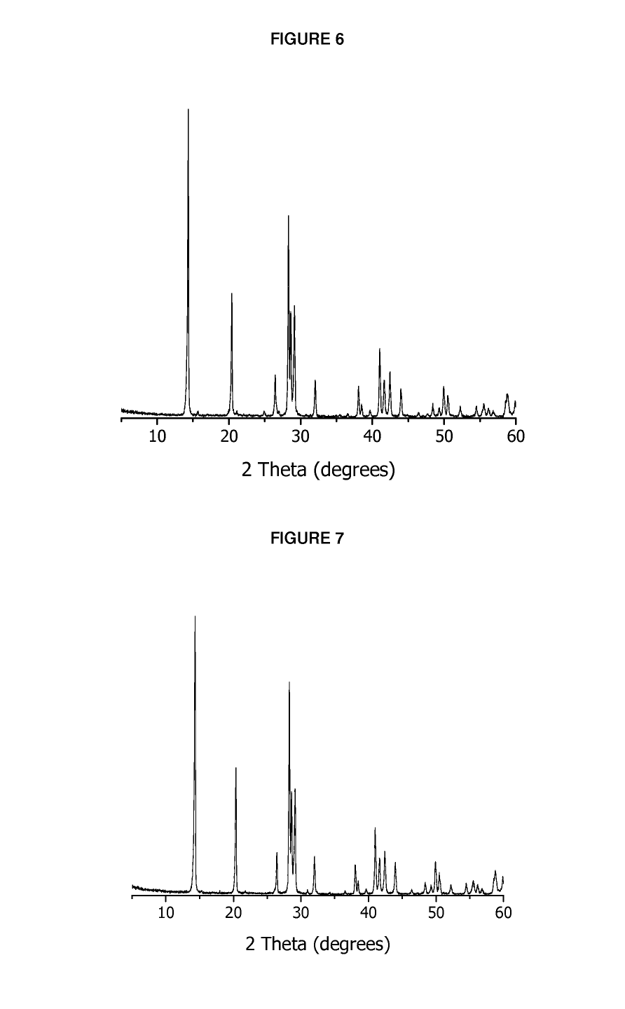

3.309 grams of lithium nitrate and 8.474 grams of ammonium heptamolybdate are dissolved in 41.1 ml of distilled water at 50° C. Then, 400 ml of distilled water and 5.615 grams of ammonium metavanadate are added to former solution, under constant stirring. Afterward, the solution is cooled down to room temperature, adding 2.65 ml of HNO₃ (70% weight) to adjust the pH at 4, and then transferred to a Teflon coated stainless-steel autoclave. The autoclave is then heated at 150° C. for 48 hours without stirring. The hydrothermally treated solution is placed in a rotoevaporator to evaporate the solvent at 100° C. for 2 hours, and recovering the solid. The resulting solid was dried at 120° C. for 12 hours, and thermally treated at 550° C. for 4 hours, at a heating rate of 3° C./min, under air flow of 60 ml/min. The X-ray pattern of this catalyst is shown in FIG. 6. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Crystalline phases were also detected with diffraction peaks located at 2θ: 15.72, 21.14, 24.94 and 27.0.

The main catalytic results of the ODH-Et obtained with the LiVMo oxide catalyst are shown in Table 2. The reaction was performed at 600° C. using an ethane/oxygen/nitrogen flow with molar ratio of 30/10/60 and a W/F°ₑₜₕₐₙₑ of 3.4 g_{cat} h/molₑₜₕₐₙₑ Prior to the catalytic activity measurement, the catalyst was pretreated with a flow of 100 ml/min of helium at 600° C. for 1 hour.

### Example 15

Preparation of a LMMO of LiVMo oxide. The preparation of the LMMO was through the following steps: 1) precursors salts are dissolved, 2) The solvent, of the precursors salts solution, is evaporated for obtaining a catalyst with the following atomic composition: LiVMo.

4.55 grams of lithium nitrate and 11.652 grams of ammonium heptamolybdate are dissolved in 56.5 ml of distilled water at 50° C. Then, 550 ml of distilled water and 5.615 grams of ammonium metavanadate are added to former solution, under constant stirring. Afterward, the solution is cooled down to room temperature, adding 3.61 ml of HNO₃ (70% weight) to adjust the pH at 4. Then, the solution is placed in a rotoevaporator to evaporate the solvent at 100° C. for 2 hours, and recovering the solid. The resulting solid was dried at 120° C. for 12 hours, and thermally treated at 550° C. for 4 hours, with a heating rate of 3° C./min, under air flow of 60 ml/min. The X-ray pattern of this catalyst is shown in FIG. 7. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Crystalline phases were also detected with diffraction peaks located at 2θ: 15.32, 18.00, 21.80, 26.14, 30.96 and 34.32.

The main catalytic results of the ODH-Et obtained with the LiVMoO catalyst are shown in Table 2. The reaction was performed at 600° C. using a using an ethane/oxygen/nitrogen flow with molar ratio of 30/10/60 and a W/F°ₑₜₕₐₙₑ of 6.8 g_{cat} h/molₑₜₕₐₙₑ. Prior to the catalytic activity measurement, the catalyst was pretreated with a flow of 100 ml/min of helium at 600° C. for 1 hour.

### Example 16

Preparation of a LMMO of LiVMo oxide. The preparation of the LMMO was through the following steps: 1) precursors salts are dissolved, 2) the solution was hydrothermally treated, and 3) The solvent, of the hydrothermally treated precursors salts solution, was evaporated to obtain a catalyst with the following atomic composition: LiVMo.

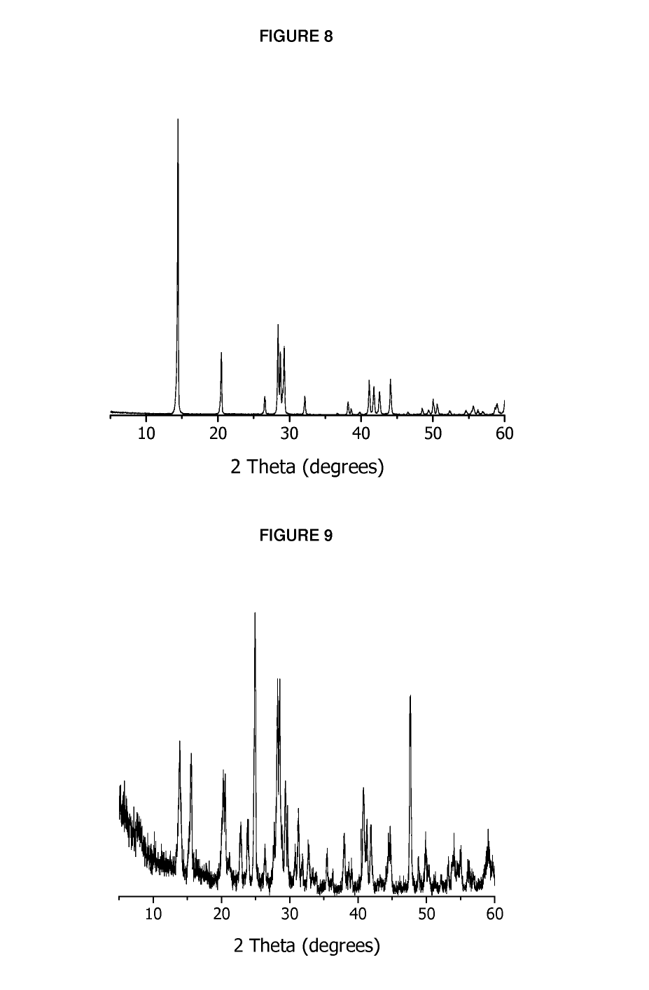

4.55 grams of lithium nitrate and 11.652 grams of ammonium heptamolybdate are dissolved in 56.5 ml of distilled water at 50° C. Then, 550 ml of distilled water and 5.615 grams of ammonium metavanadate are added to former solution, under constant stirring. Afterward, the solution is cooled down to room temperature, adding 3.61 ml of HNO₃ (70% weight) to adjust the pH at 4, and then transferred to a Teflon coated stainless-steel autoclave. The autoclave is then heated at 150° C. for 24 hours without stirring. The hydrothermally treated solution is placed in a rotoevaporator to evaporate the solvent at 100° C. for 2 hours, and recovering the solid. The resulting solid was dried at 120° C. for 12 hours, and thermally treated at 550° C. for 4 hours, with a heating rate of 3° C./min, under air flow of 60 ml/min. The X-ray pattern of this catalyst is shown in FIG. 8. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17.

The main catalytic results of the ODH-Et obtained with the LiVMo catalyst are shown in Table 2. The reaction was performed at 600° C. using a using an ethane/oxygen/nitrogen flow with molar ratio of 30/10/60 and a W/F°ₑₜₕₐₙₑ of 3.4 g_{cat} h/molₑₜₕₐₙₑ Prior to the catalytic activity measurement, the catalyst was pretreated with a flow of 100 ml/min of helium at 600° C. for 1 hour.

### Example 17

The ODH-Et reaction with the LiVMo oxide catalyst of Example 16 was performed at 600° C. using an ethane/oxygen/nitrogen flow with molar ratio of 30/10/60 and a W/F°ₑₜₕₐₙₑ of 6.8 g_{cat} h/molₑₜₕₐₙₑ. Prior to the catalytic activity measurement, the catalyst was pretreated with a flow of 100 ml/min of helium at 600° C. during 1 hour. The main catalytic results of the ODH-Et obtained with the LiVMo oxide catalyst are shown in Table 2.

### Example 18

Preparation of a LMMO of LiVMo oxide. The preparation of the LMMO was performed following the process comprising the steps of: 1) mixing the powder of the precursors salts, 2) incorporation of organic chemical agents to the mixture of precursors salts, and 3) hydrothermal treatment of the precursors salts paste for obtaining a catalyst with the following atomic composition: LiVMo oxide.

3.309 grams of lithium nitrate (LiNO₃), 5.615 grams of ammonium vanadate (NH₄VO₃) and 8.474 grams of ammonium heptamolybdate ((NH₄)₆Mo₇O₂₄.4H₂O) are mixed in an agate mortar, then this solid mixture is impregnated by incipient wetness method with a mixture of 1.5 ml of water, 1.5 ml of ethanol and 1.5 ml of hydrazine, until obtaining a brown solid paste, which is then transferred to a Teflon coated stainless-steel autoclave. Subsequently, the recovered solid from the hydrothermal treatment was dried at 120° C. for 12 hours, and thermally treated at 550° C. during 4 hours, with a heating rate of 3° C./min, under air flow of 60 ml/min. The X-ray pattern of this catalyst is shown in FIG. 9. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Crystalline phases were also detected with diffraction peaks located at 2θ: 15.60, 21.18, 22.88, 23.94, 24.94, 30.78, 31.28, 32.70, 35.50, 37.96, 40.80, 44.72, 47.60, and 48.78.

The main catalytic results of the ODH-Et obtained with the LiVMo oxide catalyst are shown in Table 2. The reaction was performed at 600° C. using a using an ethane/oxygen/nitrogen flow with molar ratio of 30/10/60 and a W/F°ₑₜₕₐₙₑ of 6.8 g_{cat} h/molₑₜₕₐₙₑ. Prior to the catalytic activity measurement, the catalyst was pretreated with a flow of 100 ml/min of helium at 600° C. for 1 hour.

### Example 19

Preparation of a LMMO of LiVMo. The preparation of the LMMO was performed following the process comprising the steps of: 1) Mixing the precursors salt powders, 2) Addition of an organic chemical agent to the former mixture for obtaining a solid paste, 3) Dissolving the solid paste mixture, and 4) a hydrothermal treatment of the solubilized solid paste to obtain a material, which can act as catalyst, with the following atomic composition: LiVMo.

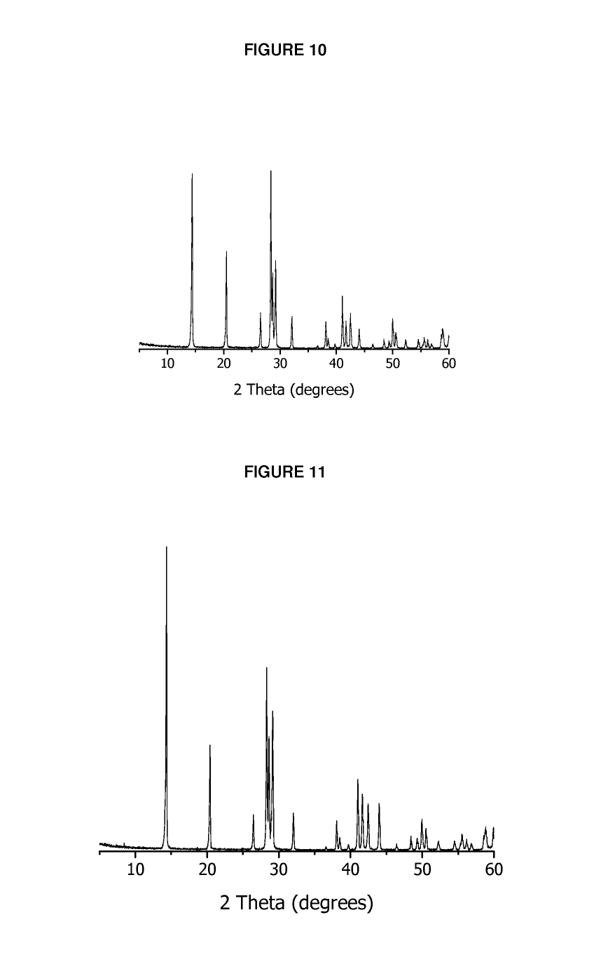

For that, 3.309 gram of lithium nitrate (LiNO₃), 5.615 grams of ammonium vanadate (NH₄VO₃) and 8.47 grams of ammonium heptamolybdate ((NH₄)₆Mo₇O₂₄.4H₂O) are mixed in an agate mortar, then this solid mixture is impregnated by incipient wetness method with a mixture of 1.5 ml of water, 1.5 ml of ethanol and 1.5 ml of hydrazine, to obtain a brown solid paste. Next, the brown solid paste is dissolved in 441 ml of distilled water at 50° C. under constant stirring during 1 hour. Then, the solution was cooled at room temperature, adding 6.67 ml of HNO₃ (70% weight) to adjust the pH at 4, and then transferred to a teflon coated stainless-steel autoclave. The autoclave is then heated at 150° C. for 48 hours without stirring. The hydrothermally treated solution is placed in a rotoevaporator to evaporate the solvent at 100° C. for 2 hours, for recovering the solid. The obtained solid was dried at 120° C. for 12 hours, and thermally treated at 550° C. for 4 hours, with a heating rate of 3° C./min, under air flow of 60 ml/min. The X-ray pattern of this catalyst is shown in FIG. 10. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17.

The main catalytic results of the ODH-Et obtained with the LiVMo oxide catalyst are shown in Table 2. The reaction was performed at 600° C. using an ethane/oxygen/nitrogen flow with molar ratio of 30/10/60 and a W/F°ₑₜₕₐₙₑ of 6.8 g_{cat} h/molₑₜₕₐₙₑ. Prior to the catalytic activity measurement, the catalyst was pretreated with a flow of 100 ml/min of helium at 600° C. for 1 hour.

### Example 20

Preparation of a LMMO of LiVMo oxide. The preparation of the LMMO was performed following the process comprising the steps of: 1) Mixing the precursors salt powders, and 2) Addition of an organic chemical agent to the former mixture for obtaining a solid paste, which can act as catalyst, with the following atomic composition LiVMo oxide.

3.309 gram of lithium nitrate (LiNO₃), 5.615 grams of ammonium vanadate (NH₄VO₃) and 8.47 grams of ammonium heptamolybdate ((NH₄)₆Mo₇O₂₄.4H₂O) are mixed in an agate mortar, then this solid mixture is impregnated by incipient wetness method with a mixture of 1.0 ml of water, 0.5 ml of ethanol and 0.5 ml of hydrazine, until obtaining a brown solid paste, which it is allowed to stand during 2 hours at room temperature. Subsequently, the recovered solid was dried at 120° C. for 12 hours, and thermally treated at 550° C. for 4 hours, with a heating rate of 3° C./min, under air flow of 60 ml/min. The X-ray pattern of this catalyst is shown in FIG. 11. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17.

The main catalytic results of the ODH-Et obtained with the LiVMo oxide catalyst are shown in Table 2. The reaction was performed at 600° C. using an ethane/oxygen/nitrogen flow with molar ratio of 30/10/60 and a W/F°ₑₜₕₐₙₑ of 6.8 g_{cat} h/molₑₜₕₐₙₑ. Prior to the catalytic activity measurement, the catalyst was pretreated with a flow of 100 ml/min of helium at 600° C. for 1 hour.

### Example 21

Preparation of a LMMO of LiVMo oxide. The preparation of the LMMO was performed following the process comprising the steps of: 1) mixing the powder of the precursors salts, and 2) incorporation of organic chemical agents to the mixture of precursors salts for obtaining a catalyst with the following atomic composition: Li_{1.2}V_{1.2}Mo_{0.8}. oxide

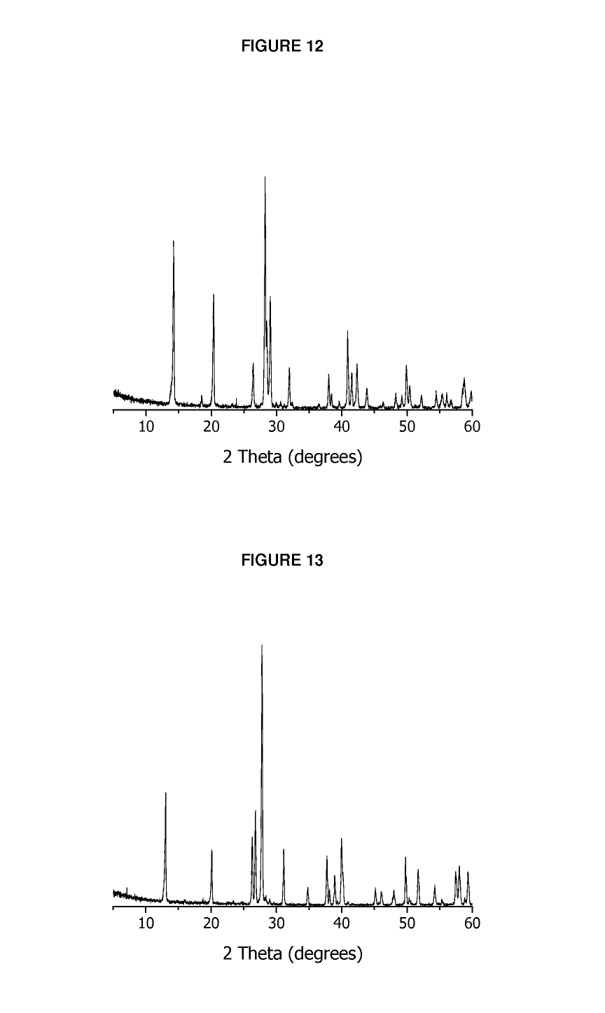

3.309 gram of lithium nitrate (LiNO₃), 5.615 grams of ammonium vanadate (NH₄VO₃) and 5.650 grams of ammonium heptamolybdate ((NH₄)₆Mo₇O₂₄.4H₂O) are mixed in an agate mortar, then this solid mixture is impregnated by incipient wetness adding a mixture of 0.9 ml of water, 0.5 ml of ethanol and 0.5 ml of hydrazine, to obtain a brown solid paste, which it is allowed to stand for 2 hours at room temperature. Subsequently, the recovered solid was dried at 120° C. for 12 hours, and thermally treated at 550° C. for 4 hours, with a heating rate of 3° C./min, under air flow of 60 ml/min. The X-ray pattern of this catalyst is shown in FIG. 12. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Crystalline phases were also detected with diffraction peaks located at 2θ: 18.52, 23.26, 30.0, 30.68, 32.40, and 48.30.

The main catalytic results of the ODH-Et obtained with the LiVMo oxide catalyst are shown in Table 2. The reaction was performed at 600° C. using an ethane/oxygen/nitrogen flow with molar ratio of 30/10/60 and a W/F°ₑₜₕₐₙₑ of 6.8 g_{cat} h/molₑₜₕₐₙₑ. Prior to the catalytic activity measurement, the catalyst was pretreated with a flow of 100 ml/min of helium at 600° C. for 1 hour.

TABLE 2
Main catalytic results of the ODH-Et for producing ethylene,
reaction test performed at 600° C., using the activated LMMO of the
present invention, examples 14 to 21.

| | | | Ethylene | |
|---|---|---|---|---|
| | | W/F°_{ethane,} | selectivity, | Ethylene |
| Example | Catalyst | g_{cat} h/molₑₜₕₐₙₑ | mol % | production^{a} |
| 14 | LiVMo | 3.4 | 98 | 646 |
| 15 | LiVMo | 6.8 | 73 | 992 |
| 16 | LiVMo | 3.4 | 84 | 1,314 |
| 17 | LiVMo | 6.8 | 71 | 1,286 |
| 18 | LiVMo | 6.8 | 63 | 1,141 |
| 19 | LiVMo | 6.8 | 62 | 1,123 |
| 20 | LiVMo | 6.8 | 68 | 1,232 |
| 21 | Li_{1.2}V_{1.2}Mo_{0.8} | 6.8 | 66 | 1,223 |

^{a}Units: grams of ethylene produced per hour per kilogram of catalyst

### Example 22

Preparation of a LMMO of NaVMo. The preparation of the LMMO was performed following the process comprising the steps of: 1) Mixing the precursors salt powders, and 2) Addition of an organic chemical agent to the former mixture for obtaining a solid paste, which can act as catalyst, with the following atomic composition: NaVMo oxide.

4.3 gram of sodium nitrate (NaNO₃), 5.8 grams of ammonium vanadate (NH₄VO₃) and 8.8 grams of ammonium heptamolybdate ((NH₄)₆Mo₇O₂₄.4H₂O) are mixed in an agate mortar, then this solid mixture is impregnated by incipient wetness method with a mixture of 2.2 ml of water, 0.5 ml of ethanol and 0.5 ml of hydrazine, to obtain a brown solid paste, which it is allowed to stand for 2 hours at room temperature. Subsequently, the recovered solid was dried at 120° C. for 12 hours, and thermally treated at 550° C. for 4 hours, with a heating rate of 3° C./min, under air flow of 60 ml/min. The X-ray pattern of this catalyst is shown in FIG. 13. XRD pattern of LMMO can be indexed to sodium vanadium molybdenum mixed oxide (ICDD-PDF 04-011-9693) with main diffraction peaks located at 2θ: 13.09, 20.14, 26.35, 26.90, 27.83, 31.15, 37.76, and 40.05. Crystalline phases were also detected with diffraction peaks located at 2θ: 18.80, 23.44, 28.40, 29.0 and 29.64.

### Example 23

Preparation of a LMMO of AgVMo oxide. The preparation of the LMMO was performed following the process comprising the steps of: 1) dissolution precursors salts, 2) incorporation of organic chemical agents to the solution of precursors salts, 3) solvent evaporation from the solution of precursors salts and organic chemical agent for obtaining a slurry, and 4) thermal treatment of the obtaining slurry at moderate and high temperature for obtaining a catalyst with the following atomic composition: AgVMo oxide.

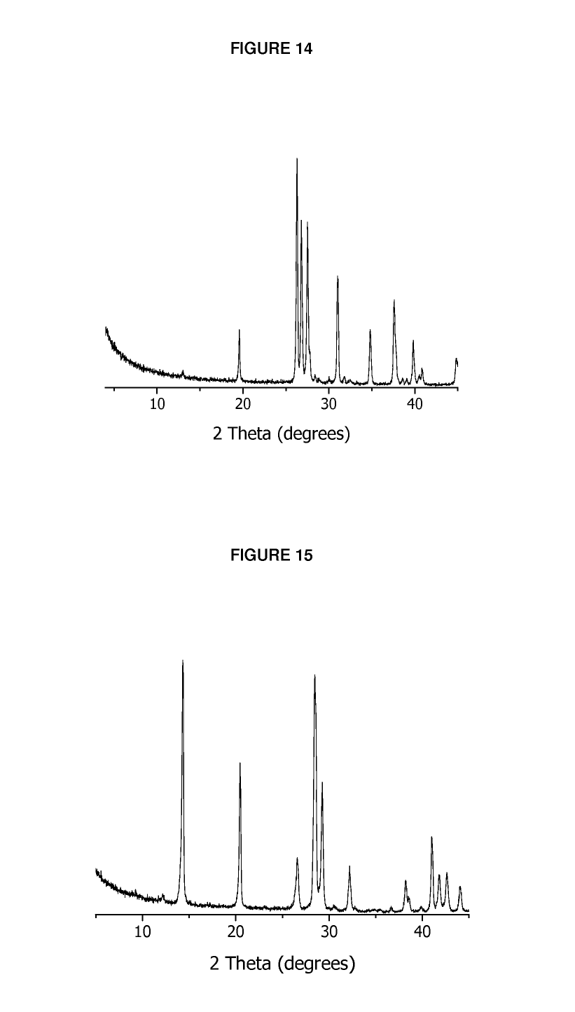

5.61 grams of silver nitrate (AgNO₃), 3.86 grams of ammonium metavanadate (NH₄VO₃) and 5.83 grams of ammonium heptamolybdate ((NH₄)₆Mo₇O₂₄.4H₂O) were dissolved in 151 ml of the distilled water at 50° C. Then, 10.62 g of glycine dissolved in 47 ml of distilled water at 50° C. is added to this solution under constant stirring. The temperature of resulting mixture is increased up to 90° C., for solvent evaporation process, until a slurry is obtained. Then, the slurry is subjected to thermal treatment at 250° C. during 30 minutes under air atmosphere to obtain a powder. The solid is then thermally treated under air atmosphere, at 300° C. for 0.5 hour and after at 550° C. for 4 hours. The X-ray pattern of this catalyst is shown in FIG. 14. XRD pattern of LMMO can be indexed to silver vanadium molybdenum mixed oxide (ICDD-PDF 04-002-4830) with main diffraction peaks located at 2θ: 19.56, 26.35, 26.77, 27.58, 31.09, 34.83, 37.58, 44.88, 49.88, and 51.54. Crystalline phases were also detected with diffraction peaks located at 2θ: 28.38, 28.86, 30.02, 31.82, 32.46 and 33.24.

### Example 24

Preparation of a LMMO of LiZnVMo oxide. The preparation of the LMMO was performed following the process comprising the steps of: 1) dissolution precursors salts, 2) incorporation of organic chemical agents to the solution of precursors salts, 3) solvent evaporation from the solution of precursors salts and organic chemical agent for obtaining a slurry, and 4) thermal treatment of the obtaining slurry at moderate and high temperature, to obtain a catalyst with the following atomic composition: Li_{0.8}Zn_{0.2}V_{1.2}Mo_{0.8} oxide.

1.96 grams of zinc nitrate (Zn(NO₃).6H₂O), 1.81 grams of lithium nitrate (LiNO₃), 4.63 of ammonium metavanadate (NH₄VO₃) and 4.66 grams of ammonium heptamolybdate ((NH₄)₈Mo₇O₂₄.4H₂O) were dissolved in 160 ml of the distilled water at 50° C., stirring constantly for 30 minutes. Then, 11.47 g of glycine dissolved in 51 ml of deionized water at 50° C. is added to this solution, under constant stirring. The temperature of resulting mixture is increased up to 90° C., for solvent evaporation process, to obtain a slurry. Then, the slurry is subjected to thermal treatment at 250° C. during 30 minutes under air atmosphere to obtain a powder. The solid is then thermally treated in a muffle on air atmosphere, at 300° C. for 0.5 hour and after at 550° C. for 4 hours. The X-ray pattern of this catalyst is shown in FIG. 15. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Crystalline phases were also detected with diffraction peaks located at 2θ: 12.18, 23.10, 30.52, and 32.78

### Example 25

Preparation of a LMMO of LiAgVMo oxide. The preparation of the LMMO was performed following the process comprising the steps of: 1) dissolution precursors salts, 2) incorporation of organic chemical agents to the solution of precursors salts, 3) solvent evaporation from the solution of precursors salts and organic chemical agent for obtaining a slurry, and 4) thermal treatment to obtain a slurry at moderate and high temperature to obtain a catalyst with the following atomic composition: Li_{0.8}Ag_{0.2}VMo oxide.

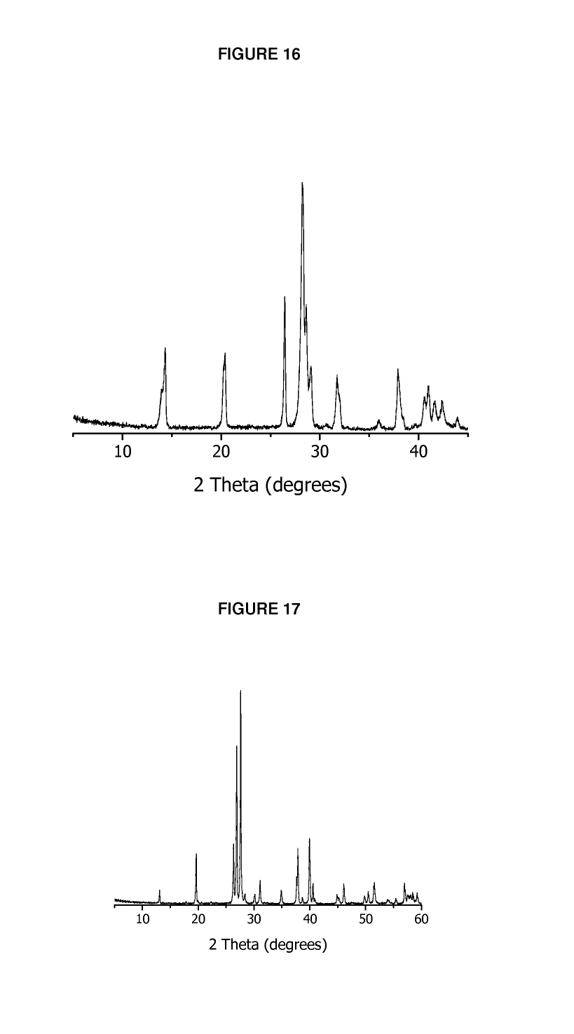

1.12 grams of silver nitrate (AgNO₃), 1.82 grams of lithium nitrate (LiNO₃), 3.86 of ammonium metavanadate (NH₄VO₃) and 5.83 grams of ammonium heptamolybdate ((NH₄)₆Mo₇O₂₄.4H₂O) are dissolved in 151.6 ml of the distilled water at 50° C. Then, 10.62 g of glycine dissolved in 47 ml of distilled water at 50° C. is added to this solution under constant stirring. The temperature of the resulting mixture is increased up to 90° C., for solvent evaporation process, to obtain a slurry. Then, the slurry is subjected to thermal treatment in an oven at 250° C. for 30 minutes under air atmosphere to obtain a powder. The solid is then thermally treated in a muffle on air atmosphere, at 300° C. for 0.5 hour and after at 550° C. for 4 hours. The X-ray pattern of this catalyst is shown in FIG. 16. XRD pattern of LMMO can be indexed to lithium vanadium molybdenum mixed oxide (ICDD-PDF 04-006-7234) with main diffraction peaks located at 2θ: 14.34, 20.48, 26.49, 28.34, 28.70, and 29.17. Crystalline phases were detected with diffraction peaks located at 2θ: 14.0, 31.76, 36.02, 37.92 and 40.64.

### Example 26

Preparation of a LMMO of AgVMo oxide. The preparation of the LMMO was performed by the solid state reaction method to obtain a catalyst with the following atomic composition: AgVMo. For that, 4.827 grams of silver nitrate (AgNO₃), 3.32 grams of ammonium metavanadate (NH₄VO₃) and 5.01 grams of ammonium heptamolybdate, (NH₄)₆Mo₇O₂₄.4H₂O, are mixed for 1 hour in an agate mortar, adding acetone to facilitate and improve the mixing and the homogeneity of the components until a yellow solid paste is obtained. Then, the paste is dried at 110° C. for 1 hour under air atmosphere, and it ground to obtain a yellow powder. The dried yellow powder is thermally treated in a muffle under air atmosphere, at 300° C. for 1 hour and after at 550° C. for 24 hours. This catalyst is characterized by an X-ray pattern shown in FIG. 17. XRD pattern of LMMO can be indexed to silver vanadium molybdenum mixed oxide (ICDD-PDF 04-002-4830) with main diffraction peaks located at 2θ: 19.56, 26.35, 26.77, 27.58, 31.09, 34.83, 37.58, 44.88, 49.88, and 51.54. Crystalline phases were also detected with diffraction peaks located at 2θ: 17.84, 19.92, 20.62, 22.34, 23.24, 28.42, 30.16, and 55.44.

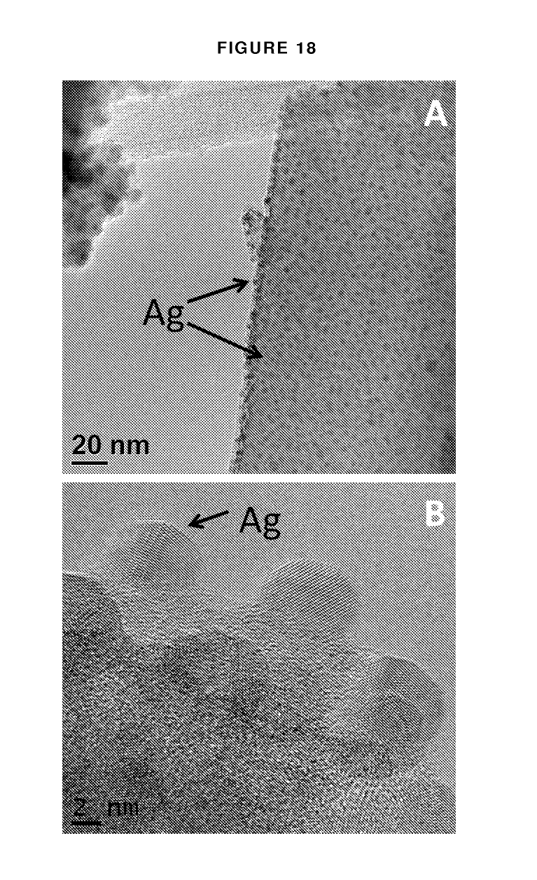

On this sample a High Resolution of Transmission Electronic Microscopy (HRTEM) study was performed. The HRTEM study is reported in FIG. 18. Image (A) shows representative catalyst's particle. It was noticed that the particle is decorated by nanometric Ag metallic particles highly dispersed, and image (B) is a close-up of the metallic particles, which were indexed as nanometric metallic silver. It is important to mention, that increasing the silver content, one portion of it is forming part of the layered structure and the excess is highly dispersed on the AgVMo particle surface.

1-13. (canceled)

14. A process for preparing a nanometer and micrometer layered multimetallic oxide catalyst having the formula
M1 M2 M3 O_{δ}
wherein:
M1 is selected from the group of Ag, Au, Zn, Sn, Rh, Pd, Pt, Cu, Ni, Fe, Co, an alkaline metal, an alkaline earth metal, a rare earth metal, and mixtures thereof;
M2 is selected from the group of Ti, Hf, Zr, Sn, Bi, Sb, V, Nb, Ta and P, and mixtures thereof;
M3 is selected from the group of Mo, W and Cr, and mixtures thereof;
and where said multilayered metallic oxide exhibits a major X-ray diffraction peak between 5<2θ<15,
said process comprising the steps of
mixing metallic precursors of M₁, M₂ and M₃ to form a precursor mixture,
hydrothermal treatment of the resulting mixture to obtain a homogeneous solid mixture, and
thermally treating the solid mixture to activate the solid mixture and obtain said catalyst.

15. The process of claim 14, wherein
the precursors are mixed by mechanical mixing or by dissolution of the corresponding metal salts.

16. The process of claim 14, further comprising
adjusting the pH of the resulting solution.

17. The process of claim 14, further comprising the steps of
adding a chemical agent to the precursor mixture selected from the group consisting of an amino acid, glycine, amines, urea or carboxylic acids, or a mixture thereof.

18. The process of claim 14, wherein
said precursors are selected from the group consisting of pure metallic elements, metallic salts, metallic oxides, metallic hydroxides, metallic alkoxides, acids, and mixtures thereof.

19. The process of claim 18, wherein
said precursors are selected from the group consisting of nitrates, oxalates, sulfates, carbonates, halides, and mixtures thereof.

20. The process of claim 14, wherein
said catalyst exhibits at least one X-ray diffraction pattern selected from the group consisting of monoclinic lattice of silver vanadium molybdenum oxide corresponding to ICDD-PDF 04-002-4830, or cesium vanadium molybdenum oxide corresponding to ICDD-PDF 00-030-0381, or monoclinic sodium vanadium molybdenum oxide corresponding to ICDD-PDF 04-011-9693, or monoclinic lithium vanadium molybdenum oxide corresponding to ICDD-PDF 04-006-7234, or orthorhombic calcium vanadium molybdenum oxide corresponding to ICDD-PDF 04-013-4035.

21. The process of claim 14, wherein
said catalyst exhibits at least one X-ray diffraction peak between 10<2θ<15.

22. The process of claim 14, further comprising
depositing said catalyst on a solid support selected from the group consisting of silica, silica-gel, amorphous silica, zirconium oxide, alumina, titanium oxide, aluminum-silicates, and mixtures thereof in an amount of 20 wt % to 70 wt % based on the total weight of the catalyst and support.

23. A process for the oxidative dehydrogenation of ethane to produce ethylene comprising the steps of:
contacting ethane or ethane mixed with an oxidizing atmosphere and/or an inert atmosphere with an activated layered multimetallic mixed oxide (LMMO) catalyst having the formula
M1 M2 M3 O_{δ}
wherein:
M1 is selected from the group of Ag, Au, Zn, Sn, Rh, Pd, Pt, Cu, Ni, Fe, Co, an alkaline metal, an alkaline earth metal, earth metal, and mixtures thereof;
M2 is selected from the group of Ti, Hf, Zr, Sn, Bi, Sb, V, Nb, Ta and P, and mixtures thereof;
M3 is selected from the group of Mo, W and Cr, and mixtures thereof;
where δ depends on the amount, oxidation state and/or valence of the components;
and where the catalyst exhibits at least one X-ray diffraction peak between 5<2θ<15;
and producing ethylene.

24. The process of claim 23, wherein
said catalyst exhibits at least one X-ray diffraction pattern selected from the group consisting of monoclinic lattice of silver vanadium molybdenum oxide corresponding to ICDD-PDF 04-002-4830, or cesium vanadium molybdenum oxide corresponding to ICDD-PDF 00-030-0381, or monoclinic sodium vanadium molybdenum oxide corresponding to ICDD-PDF 04-011-9693, or monoclinic lithium vanadium molybdenum oxide corresponding to ICDD-PDF 04-006-7234, or orthorhombic calcium vanadium molybdenum oxide corresponding to ICDD-PDF 04-013-4035.

25. The process of claim 23, wherein
the oxidizing atmosphere is selected from the group consisting of oxygen, air, CO₂ and mixtures thereof.

26. The process of claim 23, wherein
said inert atmosphere is selected from the group consisting of nitrogen, argon, helium, and mixtures thereof.

27. The process of claim 23, wherein
said ODH-Et reaction is carried out in the presence of water vapor.

28. The process of claim 23, wherein
ethylene is produced at a reaction temperature between 300 and 700° C.

29. The process of claim 23, wherein
the reaction is performed at a space time, defined as the relation between catalyst mass and the molar flow of ethane supplied to the reactor, W/F°ₑₜₕₐₙₑ, between 0.01 and 50 g_{cat} h/molₑₜₕₐₙₑ.

30. The process of claim 23, wherein
ethylene is obtained, at atmospheric pressure, at a rate of greater than or equal to 1800 grams of ethylene per hour and kg of catalyst.

31. The process of claim 23, wherein
ethylene is obtained, at atmospheric pressure, at a rate of at least 600 grams of ethylene produced per hour and kg of catalyst.
